(19) United States
(12) Patent Application Publication
(10) Pub. No.: US 2016/0239633 A1
(43) Pub. Date: Aug. 18, 2016

(54) METHOD AND SYSTEM FOR MONITORING THE DEVELOPMENT OF NEONATES

(71) Applicant: KONINKLIJKE PHILIPS N.V., Eindhoven (NL)

(72) Inventors: Jurriën Carl GOSSELINK, Lichtenvoorde (NL); Niels LAUTE, Venlo (NL); Johanna Maria DE BONT, Eindhoven (NL)

(21) Appl. No.: 15/028,720

(22) PCT Filed: Oct. 8, 2014
(86) PCT No.: PCT/IB2014/065144

§ 371 Date: Apr. 12, 2016

Related U.S. Application Data
(60) Provisional application No. 61/896,705, filed on Oct. 29, 2013.

(51) Int. Cl. G06F 19/00; A61B 5/00

(52) U.S. Cl. CPC G06F 19/3437; A61B 5/0002; G06F 19/345

(57) ABSTRACT

Systems and methods for providing an indication of the relative condition of neonatal subjects. Test datasets are obtained which describe monitored vital functions of a test subject. The test dataset is compared with one or more previously obtained model datasets describing model vital functions. Based on the comparison of the test datasets with the model datasets of vital functions a progress prediction for the development of the vital functions of the test subject may be determined. An indication of the progress prediction for the development of vital functions may be presented to a user to assist in the future handling of the test subject.

### BACKGROUND

1. Field

The present disclosure pertains to a system and method for providing an indication of the relative condition of vital functions and providing a predictive model of the development of neonatal subjects.

2. Description of the Related Art

Monitoring vital functions is known to be medically relevant to determine the health of vital functions. For example, when the vital functions are human organs, monitoring the organs to determine their condition is readily performed. In particular, the organs of neonates are monitored to ensure they continue to develop and do not encounter unnecessary complications. However, a more intuitive and system and method for monitoring the development of neonates is required.

### SUMMARY

Accordingly, one or more embodiments provide a system for providing an indication of the relative condition of neonatal subjects. The system comprises one or more computer processors configured to execute computer program modules. The computer program modules comprise an input module, a development comparison module, a development prediction module, and a presentation module. The input module is configured to obtain a test dataset which describes monitored vital functions of a neonatal test subject. The development comparison module is configured to compare the test dataset with one or more previously obtained model datasets describing vital functions of neonatal subjects. The development prediction module is configured to determine, based on the comparison between the test datasets and the model datasets, a progress prediction for the future development of the neonatal test subject. The presentation module is configured to effectuate presentation of an indication of the progress prediction.

It is yet another aspect of one or more embodiments to provide a method for providing an indication of the relative condition of neonatal subjects. The method is implemented by one or more physical computer processors. The method comprises: obtaining a test dataset which describes monitored vital functions of a neonatal test subject; comparing the test dataset with one or more previously obtained model datasets describing vital functions of neonatal subjects; determining, based on the comparison between the test datasets and the model datasets, a progress prediction for the future development of the neonatal test subject; and, effectuating presentation of an indication of the progress prediction. Optionally, the method comprises selecting individual ones of the model datasets that are similar to the test dataset, such that the progress prediction is based on the similar model datasets.

It is yet another aspect of one or more embodiments to provide a system configured to provide an indication of the relative condition of neonatal subjects. The system comprises: a means for obtaining a test dataset which describes monitored vital functions of a neonatal test subject; a means for comparing the test dataset with one or more previously obtained model datasets describing vital functions of neonatal subjects; a means for determining, based on the comparison between the test datasets and the model datasets, a progress prediction for the future development of the neonatal test subject; and, a means for effectuating presentation of an indication of the progress prediction. The system may further comprise a means for selecting individual ones of the model datasets that are similar to the test dataset, such that the progress prediction is based on the similar model datasets.

These and other aspects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of any limits.

### BRIEF DESCRIPTION OF THE DRAWINGS

FIG. 1. schematically illustrates a system for providing an indication of the relative condition of neonatal subjects, in accordance with one or more embodiments;

FIG. 2 illustrates an interface showing the development of a neonate, in accordance with one or more embodiments;

FIG. 3 illustrates an interface showing the development of a neonate, in accordance with one or more embodiments;

FIG. 4 illustrates an interface showing the development of a neonate, in accordance with one or more embodiments;

FIG. 5 illustrates a method for providing an indication of the relative condition of neonatal subjects, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

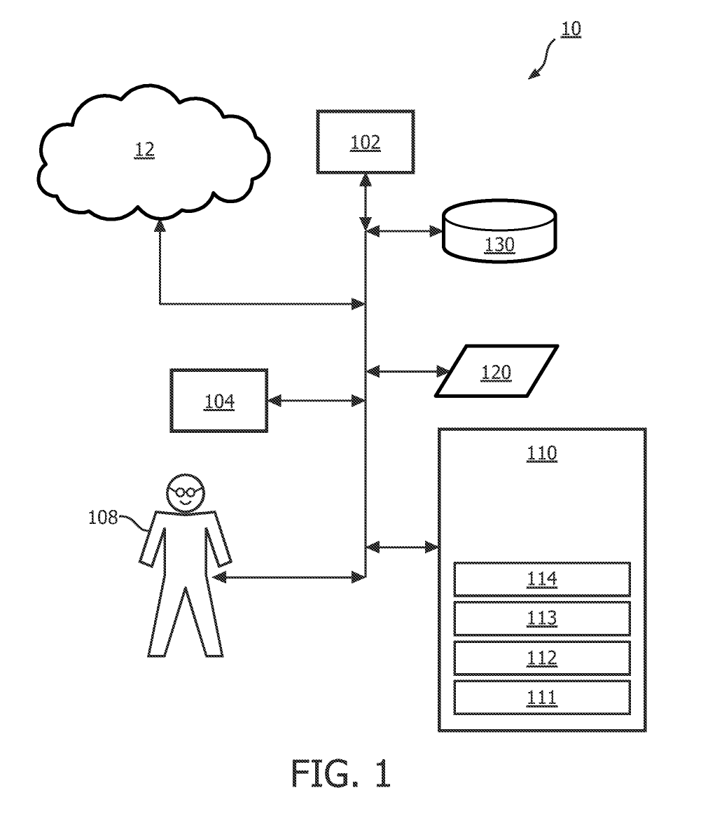

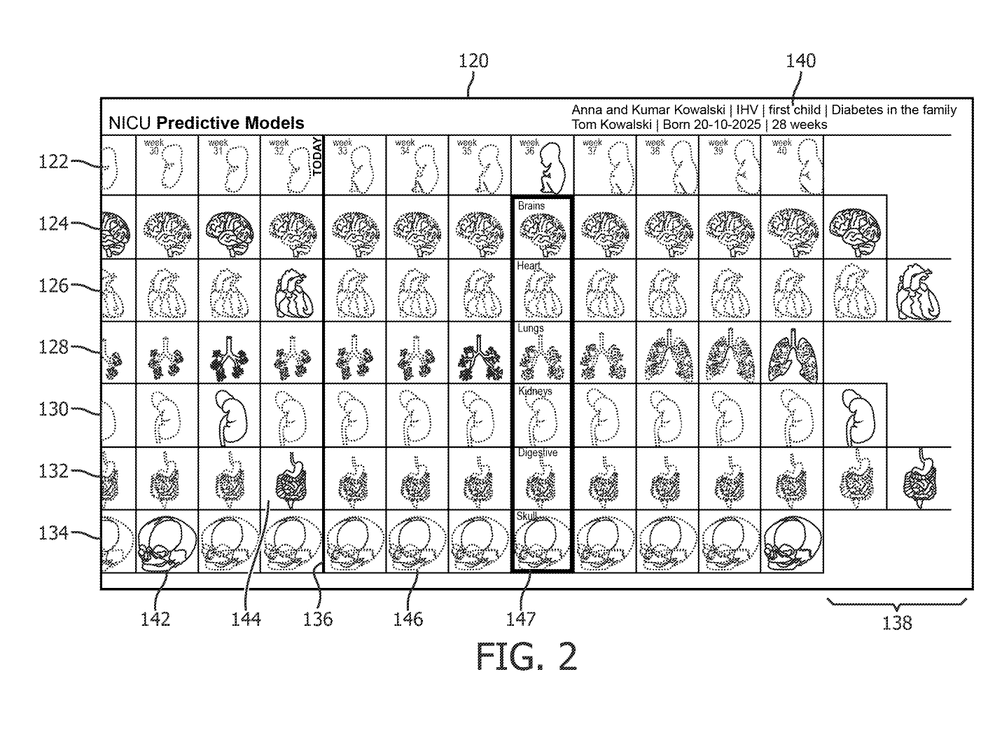

FIG. 1. schematically illustrates a system 10 for providing an indication of the relative condition of vital functions 124-134 (as shown in FIG. 2), in accordance with one or more embodiments. System 10 may interchangeably be referred to as neonatal monitoring system 10. System 10 may include one or more physical computer processors 110, electronic storage 130, user interface 120, electronic communications network 12 and/or other components and/or computer program modules. The computer program modules may include one or more of an input module 111, a development comparison module 112, a development prediction module 113, a presentation module 114, and/or other modules. Also illustrated in FIG. 1 is a user 108 of system 10 such as, by way of non-limiting example, a scientist, a care-giver, a doctor, a parent, a medical professional, and/or stake holder in the neonatal subject. Also illustrated is one or more computing platforms 102, and one or more servers 104.

Providing a data-driven predictive model for the development of a subject, for example, a developing neonate, may improve the current way decisions are made in medical care, or other area where predictive models are required. With respect to developing neonates, such predictive models may improve the current way decisions are made in Neonatal Intensive Care Units (NICU). Collecting and aggregating datasets relating to the development of subjects may improve and support decision making regarding the subjects and improve communication with, and the information provided to, patients, care givers, medical professionals, parents and other stake holders in the subject. Providing visualizations of the current and future development of the subject to caregivers, parents, and/or other decision makers, allows for better, more informed decisions to be made in relation to the subject's care.

Datasets relating to the vital functions of subjects may be obtained and aggregated to provide a database of information related to the vital functions of subjects. Information related to vital functions of subjects may include developmental information of organs and/or other vital functions, such that a database of the developmental stages of subjects is obtained. The information may be obtained by multiple medical facilities, such as hospitals, care homes, doctor's clinics, NICUs, and/or other medical facilities and aggregated on a central server or storage device. The database may be accessed across a network by medical service providers, researchers, vendors, contractors, and/or other entities having authorization to access the database.

Information relating to the vital functions of a subject, or patient, may be obtained and compared with the database. Making a comparison with the database may provide medical service providers and/or researchers an indication of the developmental status of the subject compared to the common developmental progress of a subject. For example, a comparison of a subject neonate with a database may provide medical professionals with an indication of the developmental status of the subject neonate compared to the common developmental progress of a typical neonate having the same birth time and same age. As another example, a comparison of a subject geriatric with a database containing information on geriatric subjects may provide medical professionals with an indication of the degenerative progress of the subject as compared to a typical geriatric having the similar characteristics. Having such information may assist medical providers and/or researchers to assess future care, and determine whether the subject is in worse or better condition than a typical subject. In response to this information, doctors, caregivers, scientists, parents and/or other stakeholders may modify the medical care and/or treatment for the test subject to bring the test subject's development back on track as compared with a typical subject having similar characteristics.

The aggregated information related to the vital functions of subjects in the database may provide an indication of the future development of a subject. Determining which datasets in the database most closely resemble the subject dataset may provide an indication of the future progress of development of the subject. Such information may be relevant to the future care of the subject. Medical providers and/or research scientists may be able to make a prediction on future health issues that the subject may encounter or suffer from. Maintaining medical histories of subjects until they are advanced in years may enable medical providers to make predictions of health issues that the subject may encounter throughout their lifetime.

For example, doctors, caregivers and/or scientists may be able make predictions related to the neonatal test subject's future cognitive abilities, any physical or mental disabilities the neonatal test subject may develop and/or suffer from in the future. Such information may allow the parents and/or guardians of the neonatal test subject to better plan for the future and provide an understanding, early on, of how to best care for the neonatal test subject. Additionally, it may be possible to determine, from the aggregated neonatal developmental information stored in database in electronic storage 130, treatment plans and medications to help avert or avoid future physical and/or mental issues. Additionally, having datasets related to vital functions of the neonatal test subjects, and the development of the vital functions of the neonatal test subjects over time, allows easy handover of medical information from one caregiver to the next, such as during a shift change in a NICU or to other medical practitioners when the neonatal test subject is discharged, such as a family physician or separate hospital facility.

The following description of the method and system is directed to the example of predicting the development of a subject neonate. One of ordinary skill in the art will appreciate and understand that the herein disclosed system and method may relate to the future development of a geriatric subject having deteriorating vital functions and/or health. The herein disclosed method and system may also be applied for other medical purposes, such as post-transplant monitoring and for predicting the future success or failure of an organ transplant. Additionally, the system and method may be applied to non-medical uses, such as the development of construction of a building, the progress of a project, and/or other subjects which require and/or would benefit from predictive models.

The server 102, computing platforms 104, electronic storage 130, interface 120 and/or external resources may be operatively linked via one or more electronic communication links 12. For example, such electronic communication links 12 may be established, at least in part, via a network such as the Internet and/or other networks. It will be appreciated that this is not intended to be limiting, and that the scope of this disclosure includes implementations in which servers 102, client computing platforms 104, electronic storage 130, interface 120 and/or external resources may be operatively linked via some other communication media.

Server(s) 102 may include electronic storage 130, one or more computer processors 110, and/or other components. Server 102 may include communication lines, or ports to enable the exchange of information with a network and/or other computing platforms 104. Illustration of server 102 in FIG. 1 is not intended to be limiting. The server 102 may include a plurality of hardware, software, and/or firmware components operating together to provide the functionality attributed herein to server 102. For example, server 102 may be implemented by a cloud of computing platforms operating together as server 102.

A given computing platform 104 may include one or more computer processors configured to execute computer program modules. The computer program modules may be configured to enable an expert or user associated with the given computing platform 102 to interface with system 10, external storage 130, internal storage, interface 120 and/or external resource, and/or provide other functionality attributed herein to computing platforms 104. By way of non-limiting example, the client computing platform 104 may include one or more of a desktop computer, a laptop computer, a handheld computer, a tablet computing platform, a NetBook, a Smartphone, a gaming console, and/or other computing platforms. Computing platform(s) 104 may be separate from server(s) 102. Alternatively computing platforms 104 and servers 102 may be in the same physical location and share resources as the same machine. Alternatively, servers 102 and computing platforms 104 may be located in different physical locations but may be considered the same machine.

Computer processor(s) 110 is configured to provide information processing capabilities in server 102, computing platform 104, and/or other computing device. As such, computer processor 110 may include one or more of a digital processor, an analog processor, a digital circuit designed to process information, an analog circuit designed to process information, a state machine, and/or other mechanisms for electronically processing information. Although computer processor 110 is shown in FIG. 1 as a single entity, this is for illustrative purposes only. In some implementations, computer processor 110 may include a plurality of processing units. These processing units may be physically located within the same device, or computer processor 110 may represent processing functionality of a plurality of devices operating in coordination, such as server 102 and computing platform 104. Computer processor 110 may be configured to execute modules 111-114 and/or other modules. Computer processor 110 may be configured to execute modules 111-114 by software; hardware; firmware; some combination of software, hardware, and/or firmware; and/or other mechanisms for configuring processing capabilities on computer processor 110.

It should be appreciated that although modules 111-114 are illustrated in FIG. 1 as being co-located within a single processing unit, in implementations in which computer processor 110 includes multiple processing units, such as in server 102 and computing platforms 104, one or more of modules 111-114 and/or other modules may be located remotely from the other modules. The description of the functionality provided by the different modules 111-114 described below is for illustrative purposes, and is not intended to be limiting, as any of modules 111-114 and/or other modules may provide more or less functionality than is described. For example, one or more of modules 111-114 may be eliminated, and some or all of its functionality may be provided by other ones of modules 111-114 and/or other modules. As another example, computer processor 110 may be configured to execute one or more additional modules that may perform some or all of the functionality attributed below to one of modules 111-114.

System 10 of FIG. 1 may include electronic storage 130 comprising electronic storage media that electronically stores information. The electronic storage media of electronic storage 130 includes one or both of system storage that is provided integrally (i.e., substantially non-removable) with system 10 and/or removable storage that is connectable to system 10 via, for example, a port (e.g., a USB port, a FireWire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 stores software algorithms, information determined by computer processor 110, information received via user interface 120, and/or other information that enables system 10 to function properly. For example, electronic storage 130 may record or store (a set of) one or more temperatures and/or parameters derived from output signals measured (e.g. over time) by one or more sensors (as discussed elsewhere herein), and/or other information. Electronic storage 130 may be a separate component within system 10, or electronic storage 130 may be provided integrally with one or more other components of system 10 (e.g., computer processor 110).

System 10 may include user interface 120 configured to provide an interface between system 10 and a user (e.g., user 108, a caregiver, a therapy decision-maker, etc.) through which the user can provide information to and receive information from system 10. This enables data, results, and/or instructions and any other communicable items, collectively referred to as “information,” to be communicated between the user and system 10. Examples of interface devices suitable for inclusion in user interface 120 include a keypad, buttons, switches, a keyboard, knobs, levers, a display screen, a touch screen, speakers, a microphone, an indicator light, an audible alarm, and a printer. Information may e.g. be provided to user 108 by user interface 120 in the form of auditory signals, visual signals, tactile signals, and/or other sensory signals.

By way of non-limiting example, in certain embodiments, user interface 120 includes a radiation source capable of emitting light. The radiation source includes one or more of an LED, a light bulb, a display screen, and/or other sources. User interface 120 may control the radiation source to emit light in a manner that conveys information to, e.g., user 108 related to, e.g., the development of the vital functions of a neonate.

It is to be understood that other communication techniques, either hard-wired or wireless, are also contemplated herein as user interface 120. For example, in one embodiment, user interface 120 is integrated with a removable storage interface provided by electronic storage 130. In this example, information is loaded into system 10 from removable storage (e.g., a smart card, a flash drive, a removable disk, etc.) that enables the user(s) to customize the implementation of system 10. Other exemplary input devices and techniques adapted for use with system 10 as user interface 120 include, but are not limited to, an RS-232 port, RF link, an IR link, modem (telephone, cable, Ethernet, internet or other). In short, any technique for communicating information with system 10 is contemplated as user interface 120.

Input module 111 is configured to obtain a test dataset which describes monitored vital functions of a neonatal test subject. The test dataset may be obtained from measurements taken by medical professionals and/or caregivers of the neonatal test subject. Information included in the test dataset may also be obtained from testimony of observations from medical professionals, caregivers, parents and/or stakeholders in the neonatal test subject. Information for the test dataset may be entered into system 10 using one or more computing platforms 102 and/or servers 104. Measuring devices used to monitor vital functions 124-134 (as shown in FIG. 2) of the neonatal test subject may be electronically connected to system 10 such that readings and/or measurements taken by the measuring devices feed information to system 10. Input module 111 may be configured to obtain a test dataset from the measuring devices.

The test dataset may comprise information related to the current condition of the vital functions of the neonatal test subject. The test dataset may comprise time-based information related to the condition of the vital functions of the neonatal test subject over a period of time. Such periods of time may be an hour, a day, a week, a month, and/or any other period of time. Information related to the vital functions of the neonatal test subject may include information from conception up to the present day. Information related to the vital functions of the neonatal test subject may also include information related to predictive future developments of the neonatal test subject.

Development comparison module 112 is configured to compare the test dataset with one or more previously obtained model datasets describing vital functions of neonatal subjects. The one or more previously obtained model datasets may describe the vital functions of neonatal subjects previously entered into and/or monitored by system 10. The model datasets may describe the development and/or condition of vital functions 124-134 of other neonates over a period of time. The period of time may include any period of time, including, but not limited to, from conception through to adulthood and beyond, including birth, childhood and other time periods. Included in the model datasets may be information related to medical and/or developmental milestones and conditions of vital functions 124-134 for each of the model neonatal subjects. For example, medical conditions appearing in childhood or during adulthood may be included in the model datasets. The model datasets may be grouped and/or categorized based on one or more parameters of the model datasets. For example, model datasets may be grouped by week of birth during the gestation period, i.e. those neonates born during the 28^{th} week of gestation may be grouped together, and those born during the 34^{th} week may be grouped together.

Development comparison module 112 may be configured to obtain the previously obtained model datasets from one or more databases of datasets of neonatal subjects. One or more centralized databases may be maintained and configured to store datasets of the vital functions of neonatal subjects. For example, the one or more databases may be stored in electronic storage 130. Computing devices 102 may be linked over one or more electronic communication networks 12 with electronic storage 130 and may be configured to access the model datasets stored in electronic storage 130. In other embodiments, model datasets may be stored locally at client device 102 or at server 104. For example, medical facilities and/or NICUs may have server(s) 104 configured to support multiple client devices 102 within the facility. In other embodiments, computing device(s) 102 and/or servers 104 from multiple medical facilities may be supported by a database stored at one location in electronic storage 130, such as a location operated by the provider of system 10.

Development comparison module 112 may be further configured to select individual ones of the model datasets that are similar to the test dataset. Factors dictating similarities between datasets may include the time of birth of the neonates in the gestation period, the weight of the neonate at birth, the level of current development of vital functions 124-134 of the neonate, specific combination of the levels of development for each of the monitored vital functions 124-134 at the current time, the specific developmental progress of the vital functions 124-134 of the neonate over time, one or more conditions of vital functions 124-134 of the neonate, one or more mental or physical conditions of the neonate, family medical history and/or other factors related to the development of the neonate.

Each similar dataset may provide an indication of the future development of the neonatal test subject. The closer the profile of the similar datasets are to the test dataset the more likely the neonatal test subject will follow a development path predicted by those similar datasets. Development comparison module 112 may be further configured to assign a weighting to the selected similar model datasets based on their closeness to the test dataset. The closer the model dataset to the test dataset the higher the weighting.

Development prediction module 113 is configured to determine, based on the comparison between the test datasets and the model datasets, a progress prediction for the future development of the neonatal test subject. Development prediction module 113 may be further configured to determine the progress prediction of vital functions 124-134 of the neonatal test subject based on the similar model datasets. Development prediction module 113 may be configured to determine the progress prediction based on the weighting of the selected similar model dataset.

Presentation module 114 may be configured to effectuate presentation of an indication of the progress prediction. Presentation of an indication of the progress prediction may be effectuated on interface 120. Interface 120 may be integrated into computing platform 104. Interface 120 may be separate from computing platform 104 but electronically linked to relay information between interface 120 and computing platform 104. Interface 120 may be an interactive interface, such that user 108 may control system 10 using interface 120. Interface 120 may be configured to be intuitively used by medical practitioners and caregivers. Interface 120 may be configured to assist medical practitioners and caregivers to explain the development predictions for vital functions 124-134 of the neonatal test subject.

Interface 120 may be configured to accept selections and/or entries by user 108 to enter information related to the neonatal test subject as well as manipulate interface 120 to modify views and select different viewable elements.

Presentation module 114 may be further configured to effectuate presentation of a notification when the comparison of the test dataset and the model datasets indicates that the neonatal test subject exceeds a development threshold from the model neonate. For example, as described herein, development comparison module 112 and/or development prediction module 114 may be configured to determine when the development levels of vital organs 124-134 of the neonatal test subject exceeds a development threshold from the model neonate. Such notifications may be graduated. For example there may be multiple levels of thresholds each having a unique notification. For example, should the test dataset indicate that the development level of any of the vital organs 124-134 of the neonatal test subject are underdeveloped by one week compared to model datasets, an amber or orange indication may be presented. For example there may be multiple levels of thresholds each having a unique notification. For example, should the test dataset indicate that the development level of any of the vital organs 124-134 of the neonatal test subject are underdeveloped by one week compared to model datasets, an amber or orange indication may be presented. As another example, should the test dataset indicate that the development level of any of the vital organs 124-134 of the neonatal test subject are underdeveloped by two or more weeks compared to model datasets, a red indication may be presented. Similarly, if the aggregated, or average, development level of vital organs 124-134 exceed a threshold from the model datasets, a further indication may be effectuated by presentation module 114.

Referring to FIG. 2, illustrated is interface 120 for visually presenting the development of the vital functions of a neonatal subject. Interface 120 may be presented on any type of graphical display device, such as a computer screen, a laptop, a tablet, a smart phone, through a projector, and/or any other graphical display device. An image of interface 120, and the information contained therein, may be presented on paper. Interface 120 may include an indication of the progress of development of model neonate 122 over time. The time period displayed may span to a period prior to the present day 136, and may extend beyond the present day to the end of a normal gestation period, i.e. 40 weeks, or even beyond. Interface 120 may visually present the development of one or more vital functions of a neonate, such as brain 124, heart 126, lungs 128, kidneys 130, gastrointestinal system 132, skull 134 and/or other vital functions. The displayed vital functions may be the monitored vital functions of the neonatal test subject.

Interface 120 may comprise an array of vital function panels 142, such that each vital function panel 142 represents the developmental level of the vital function it represents for a single time. The selected time period may be a day, a week, a month, a year, or any other time period. Each vital function panel 142, represents the development level of the vital function during a single period at a point before and/or after birth of the neonatal test subject. Each time period may be assigned a column in interface 120. The vital function panel 142 corresponding to a time period occurring at a particular point in time before and/or after the birth of the neonatal test subject may be placed in the corresponding column under model neonate 122 row for that time period. For example, vital function panel 142 represents the development of the scull at week 30 of the gestation period. Vital function panel 142 is therefore disposed under week 30 for model neonate row 122. Also, vital function panel 144 represents the development of the gastrointestinal tract at week 32 of the gestation period, and is placed in the column representing week 32, under the model neonate row 122.

Interface 120 may provide subject information 140 about the neonatal test subject. Such information may include the name of the parents, the name of the neonate, the birth date of the neonate, the week of gestation at which the neonate was born, family medical history and/or other information about the test subject or related members of the test subject's family.

Interface 120 may provide an indication of the development delay 138 of each of vital functions 124-134. Vital functions 124-134 that are developmentally behind compared to the selected model dataset 122 are indicated by a development delay period 138 extending beyond the normal gestation period. For example, in the representation illustrated in FIG. 2, lungs 128 and skull 134 have a current developmental period matching that of model neonate 122, i.e. lungs 128 and skull 134 will have the same level of development as would have occurred had the neonatal test subject remained in utero during the full gestation term of 40 weeks. However, brain 124 and kidneys 130 have a delayed development of one period, in this case one week. Similarly, the heart and gastrointestinal system have a delayed development of two periods, in this case two weeks. When the full gestation period of 40 weeks is met, brain 124, heart 126, kidneys 130, and gastrointestinal system 132 will not be at the same stage of development had they would have been if the neonatal test subject remained in utero during the full 40 week gestation term. Delayed development period 138 may be the predicted delayed development of vital functions 124-134 based on recent or the latest measurements of the monitored vital functions of the neonatal test subjects. Individual ones of vital functions 124-134 may develop at a quicker rate than expected, and others may develop at a slower rate than expected for the test subject being monitored.

Interface 120 may provide an indication of when the measurements of vital functions 124-134 were taken. For example, vital function panel 142 representing skull 134 may represent a period at which the developmental level, condition, general health, and/or other factors related to skull 134 were measured. By way of non-limiting example, such indications that a measurement of the vital function was taken during a period may include a crisper image, as opposed to a blurred image, of the vital function, such as skill 134. Such indications may also include a color, where the color indicates measurements of the vital function were monitored during that period. For example, skull 134 may have been measured during week 30 of the gestational period and therefore is represented as a clear image. Conversely, at vital function panel aligned under week 31 for the skull is blurred, indicating that the measurements of the skull were not taken during that time period.

Interface 120 may provide an indication of when vital functions 124-134 reached or are likely to reach a threshold level of development. Such an indication may be any visual indication, such as a flag, a different color, a clear image of the individual vital function as opposed to a blurred one, and/or any other indication.

Interface 120 may be configured to facilitate interactions with a user 108 of the system 10. Presentation module 114, may be configured to facilitate the selection and/or entry of one of more inputs from user 108 through interface 120. For example, user 108 may select and/or enter inputs indicating that the user wishes to more closely view the developmental level of vital functions 124-134 at week 36. User 108 may select a period 147 to view more closely. User 108 may interact with interface 120 such that user 108 selects period 147. System 10 may be configured to facilitate the display of information related to vital functions 124-134 at the 36 week period in response to an input from user 108. Period 147, as shown in FIG. 2, is four weeks into the future from the present day, indicated by present day indicator 136. System 10, therefore, may be configured to provide progress prediction information for vital functions 124-134, of the neonatal test subject, at period 147, representing week 36 of the gestation term.

The number of vital functions 124-134 illustrated in FIG. 2 is exemplary and not intended to be limited. Interface 120 may be configured to display any number of vital functions. For example, interface 120 may be configured to facilitate the navigation between vital functions 124-134 through an input device. For example, the input device may be a keyboard where user 108 may enter commands to view vital functions, the input device may be a mouse where user 108 interacts with the mouse input to move a cursor dynamically displayed on interface 120 to view vital functions, in other embodiments, the graphical display device may be a touchscreen device, such that user 108 may select virtual functions by pressing a representative area of the screen, or scroll through vital functions or time periods by swiping a finger across the screen. For example, in the representation depicted in FIG. 2, additional vital functions may be positioned in the array under skull 134. User 108 may interact with a keyboard, mouse, touchscreen, or other input device to move the view depicted on interface 120 so that user 108 can see the one or more other vital functions. Similarly, user 108 may interact with a keyboard, mouse, touchscreen, or other input device to move the view depicted on interface 120 to the left or right to view different time periods.

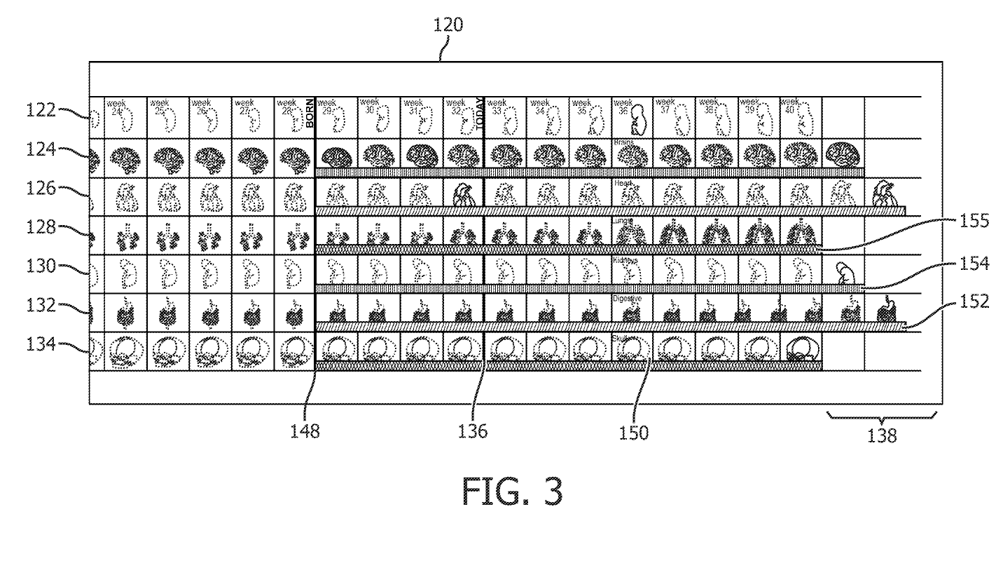

Referring now to FIG. 3, illustrated is interface 120 showing the development of a neonate, in accordance with one or more embodiments. System 10 may be configured to provide additional notifications with respect to the developmental delay of vital functions 124-134 compared to model vital function dataset 122. For example, presentation module 114 may be configured to provide notifications 152,154,155 indicating levels of developmental delay for one or more vital functions 124-134. Such indications may be color-coded, for example, a green color may be provided for vital functions having a development level within a first threshold of the model vital function dataset for a neonatal subject, an amber color may be provided for vital functions having a development level which exceeds a first threshold but are within a second threshold from the model vital function dataset, and a red color may be provided for vital functions which exceed the second threshold. As shown in FIG. 3, gastrointestinal vital function 132 has a developmental delay 138 of two periods, in this case two weeks, compared to the model vital function dataset 122. As such, for gastrointestinal vital function 132, presentation module 114 may be configured to provide an indication 152 notifying user 108 that gastrointestinal vital function 132 has a level of development exceeding a second threshold from the model vital function dataset 122, for example a red ribbon. However, for the same neonatal test subject, kidneys 130 have a developmental delay 138 of one week compared to the model vital function dataset 122. As such, for kidneys 130, presentation module 114 may be configured to provide an indication 154 notifying user 108 that kidneys 130 have a level of development exceeding a first threshold, but not a second threshold, from the model vital function dataset 122, for example an amber ribbon. Additionally, lungs 128 have no developmental delay 138 compared to the model vital function dataset 122. As such, for lungs 128, presentation module 114 may be configured to provide an indication 155 notifying user 108 that lungs 128 have a level of development not exceeding a first threshold from the model vital function dataset 122, for example a green ribbon.

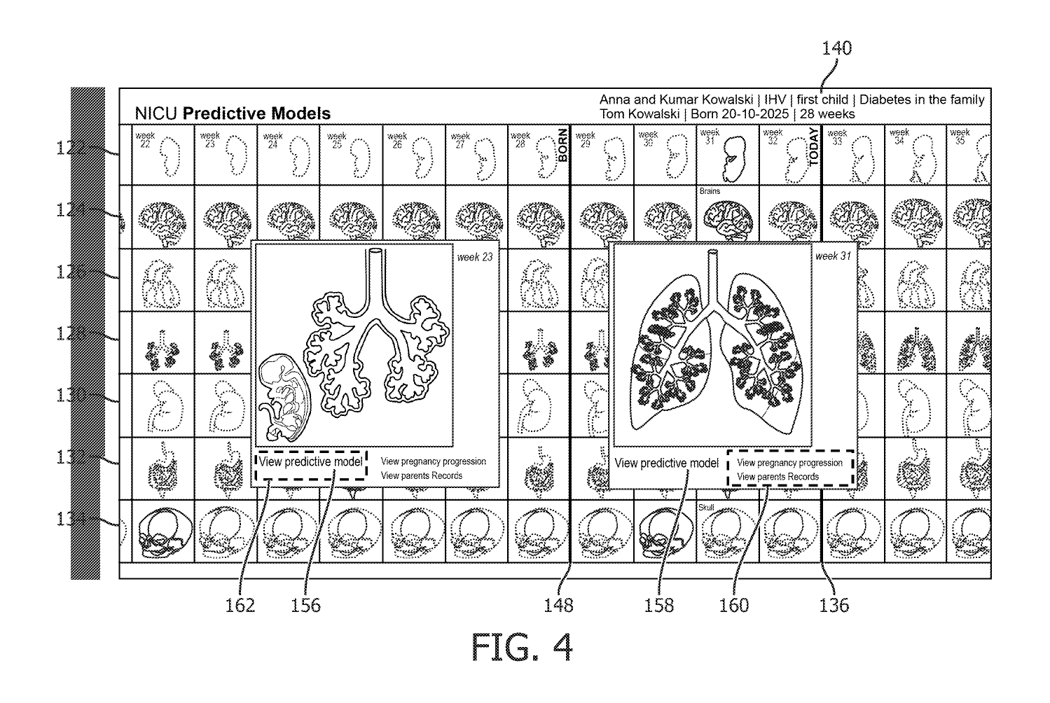

Referring to FIG. 4, illustrated is interface 120 showing the development of a neonate, in accordance with one or more embodiments. System 10 may be configured to receive entry and/or selection of an input by user 108. Such input may be entered and/or selected through a graphical user interface such as interface 120. The database, stored in electronic storage 130, may further comprise additional information related to vital functions 124-134. Additional information may include one or more specific conditions of the vital functions, or may include increased information with respect to the progress prediction for the future development of the neonatal test subject.

User 108 may enter and/or select a vital function panel, such as vital function panel 150, shown in FIG. 3. User 108 may enter and/or select vital function panel 150 by interacting with a graphical user interface, such as interface 120, at or near the location of vital function panel 150. For example, a user input device, such as a computer mouse, or keyboard, may be configured to move a cursor, or notification of selection, to vital function panel 150. As another example, a user input device, such as a touchscreen may be configured to facilitate selection and/or entry of vital function panel 150 by user 108, such that user 108 may interact with interface 120 at or near the location of vital function panel 150 on the screen of interface 120.

In response to selection and/or entry of vital function panel 150, presentation module 114 may be configured to effectuate presentation of additional information related to one or more vital functions 123-134. For example, as shown in FIG. 4, detailed information 156, 158 is shown related to lungs 128. Subsequent to an interaction with interface 120 at a vital function panel representing lungs 128 at week 23 of the gestation period, presentation module 114 may be configured to effectuate presentation of detailed information panel 156 related to lungs 128 at week 23 of the gestation period. Detailed information panel 156 may present information related to the lungs of a model neonatal subject or may present information related to the lungs of the test neonatal subject at week 23 of the gestation period. As shown in FIG. 4, week 23 of the gestation period for the test subject identified at subject information panel 140 occurred prior to birth of the neonatal test subject, indicated by birth line 148. Detailed information panel 156 related to lungs 128 at week 23 of the gestation period may further facilitate entry and/or selection of a command by user 108 to view additional information related to the neonatal test subject and/or the neonatal model subject. Additional information available to user 108 may include predictive model 162. When user 108 interacts with interface 120 to enter and/or select to view predictive model at electronic interactive button 162, presentation module 114 may be configured to effectuate presentation of the progress prediction for the development of the vital functions of the neonatal test subject from that gestational period. For example, upon interaction with electronic interactive button 162, a development progress prediction for lungs 128 from week 23 of the gestation period, onward, may be shown. The development progress prediction for lungs 128 may then be compared to the actual progress of the development of lungs 128 for the neonatal test subject identified at subject identification panel 140. The comparison between the progress prediction model and the actual progress of lungs 128 may assist caregivers, doctors, scientists, subjects, patients, and/or other stakeholders in the neonatal test subject to make decision regarding the neonatal test subjects medical care, and may provide information regarding the neonatal test subject's likely future outcome or prognosis.

Upon an interaction with a vital function panel related to lungs 128 at the 31 week gestational period, additional information 158 may be presented about the neonatal test subject's lungs during that time period. A visual comparison between the development level of lungs 128 at the 23 week gestational period and the 31 week gestation period may be made. If the detailed information 158 related to a vital function which occurs after the present day, indicated by present day line 136 on interface 120, the detailed information shown may relate to a prediction of the developmental level of the neonatal test subject at that future date. The prediction of the developmental level of the neonatal test subject may be based on the progress prediction for the future development of the neonatal subject as determined by development prediction module 113. Other information may be available upon an interaction with interface 120, either through the main interface screen, or through detailed information panels 156, 158. Other information may relate to pregnancy progression information or parental medical records. Other information may be presented to user 108 upon an interaction with electronic interactive button 160, or similar mechanism to access the other information. Other information, such as parent medical records and/or pregnancy progression information may be presented using the same interface 120 or application, or electronic interactive button 160 may be configured to facilitate the execution of one or more other applications to be displayed on one or more display devices related to system 10.

While the system and method has been described for use with neonatal test subjects, the system has broader application. The scope of this disclosure also covers degenerating vital functions as well as improving vital functions. System 10 may be used to monitor and predict the future development of geriatric subjects, wherein the future development of geriatric subjects likely includes the degeneration of vital functions and general health of the geriatric subject. For example, interface 120 may provide a visual indication of the degeneration of vital functions, or organs, during the later stages of a person's life. Predictions on when death may occur may be provided by system 10. Additionally, predictions on ailments, based on the level of degeneration of one or more vital functions 124-134 may be provided and measures may be taken to avoid those ailments. It is also contemplated that system 10 may be used after organ transplants. Model datasets may be obtained and compared against the test subject's datasets describing the transplanted vital functions. In such situations, the datasets may include factors such as lifestyle, eating habits, patient compliance information, the condition of the organ that was transplanted and/or other information useful to determine the successful acceptance of a transplanted organ. Similarly, as with geriatric and neonatal subjects, system 10, for use with transplant patients, may be configured to provide predictions on future ailments and medical problems based on the current level of development of the vital functions since transplant.

System 10 may also be used outside of the medical field for uses such as project management, when vital functions may include elements of a building being constructed, or stages of a project, such as development of a software suite, is being performed.

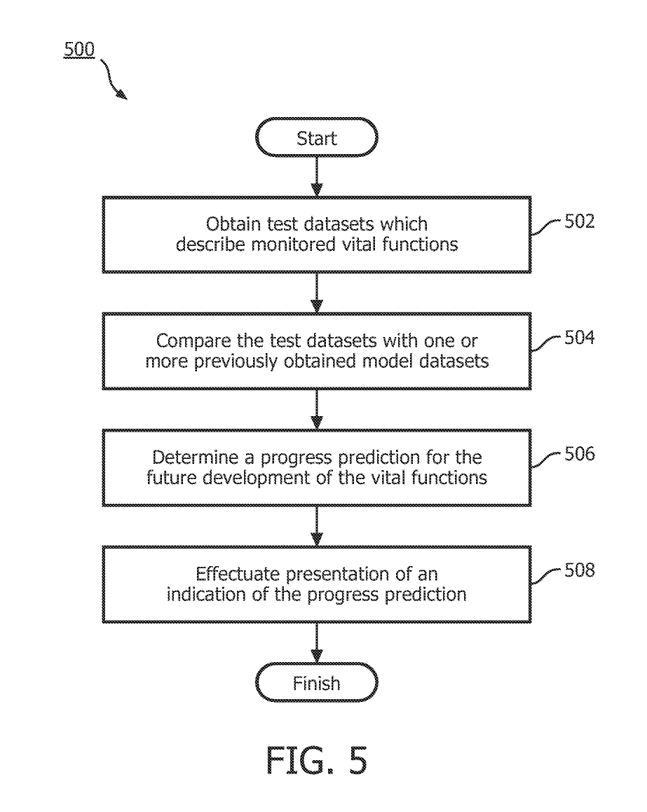

FIG. 5 illustrates a method 500 to provide an indication of the relative conditions of vital functions of a subject. The operations of method 500 presented below are intended to be illustrative. In certain embodiments, method 800 may be accomplished with one or more additional operations not described, and/or without one or more of the operations discussed. Additionally, the order in which the operations of method 500 are illustrated in FIG. 5 and described below is not intended to be limiting.

In certain embodiments, method 500 may be implemented in one or more processing devices (e.g., a digital processor, an analog processor, a digital circuit designed to process information, an analog circuit designed to process information, and/or other mechanisms for electronically processing information). The one or more processing devices may include one or more devices executing some or all of the operations of method 500 in response to instructions stored electronically on an electronic storage medium. The one or more processing devices may include one or more devices configured through hardware, firmware, and/or software to be specifically designed for execution of one or more of the operations of method 500.

At an operation 502, a test dataset may be obtained which describes monitored vital functions of a neonatal test subject. Operation 502 may be performed by an input module, such as input module 111 (shown in FIG. 1).

At an operation 504, the test dataset may be compared with one or more previously obtained model datasets describing vital functions of neonatal subjects. Individual ones of the model datasets that are similar to the test dataset may be determined and selected to compare with the test dataset. A weighting to the selected similar model datasets based on their closeness to the test dataset may be assigned. Operation 504 may be performed by a development comparison module, such as development comparison module 112 (shown in FIG. 1).

At an operation 506, based on the comparison between the test datasets and the model datasets at operation 504, a progress prediction for the future development of the neonatal test subject may be determined. The progress prediction may be based on the similar model datasets determined at operation 504. The progress prediction may be based on the weighting of the similar model dataset as assigned at operation 504. Operation 506 may be performed by a development prediction module, such as development prediction module 114 (shown in FIG. 1).

At an operation 508, presentation of an indication of the progress prediction may be effectuated. Presentation of an indication of a model dataset describing vital functions of a model neonate may be effectuated to provide a visual comparison between the vital functions of the neonatal test subject and the model neonate, where the model neonate may comprise an aggregation of information from one or more model datasets. Presentation of a notification may be effectuated in response to the comparison of the test dataset and the model datasets indicating that the neonatal test subject exceeds a development threshold from the model neonate. Operation 508 may be performed by a presentation module, such as presentation module 114 (shown in FIG. 1).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although this description includes details for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that, to the extent possible, one or more features of any embodiment are contemplated to be combined with one or more features of any other embodiment.

1. A system for providing an indication of the relative condition of neonatal subjects, the system comprising:
one or more computer processors configured to execute computer program modules, the computer program modules comprising:
an input module configured to obtain a test dataset which describes monitored vital functions of a neonatal test subject;
a development comparison module configured to compare the test dataset with one or more previously obtained model datasets describing vital functions of neonatal subjects and select, based on the comparison, individual ones of the one or more model datasets that are similar to the test dataset;
a development prediction module configured to determine, based on the test dataset and the selected model datasets, a progress prediction for the future development of the neonatal test subject; and,
a presentation module configured to effectuate presentation of an indication of the progress prediction.

2. (canceled)

3. The system of claim 1, wherein the development comparison module is further configured to assign a weighting to the selected similar model datasets based on their closeness to the test dataset, and wherein the development prediction module is further configured to determine the progress prediction based on the weighting of the selected similar model dataset.

4. The system of claim 1, wherein the presentation module is further configured to effectuate presentation of an indication of a model dataset describing vital functions of a model neonate, such that a visual comparison between the vital functions of the neonatal test subject and the model neonate is provided.

5. The system of claim 4, wherein the presentation module is further configured to effectuate presentation of a notification when a comparison of the test dataset and the selected model datasets indicates that the neonatal test subject breaches a development threshold from the model neonate.

6. A method for providing an indication of the relative condition of neonatal subjects, the method implemented by one or more physical computer processors, the method comprising:
obtaining a test dataset which describes monitored vital functions of a neonatal test subject;
comparing the test dataset with one or more previously obtained model datasets describing vital functions of neonatal subjects;
selecting, based on the comparison, individual ones of the one or more model datasets that are similar to the test dataset;
determining, based on the test dataset and the selected model datasets, a progress prediction for the future development of the neonatal test subject; and,
effectuating presentation of an indication of the progress prediction.

7. (canceled)

8. The method of claim 6, further comprising:
assigning a weighting to the selected similar model datasets based on their closeness to the test dataset; and,
determining the progress prediction is based on the weighting of the similar model dataset.

9. The method of claim 6, further comprising effectuating presentation of an indication of a model dataset describing vital functions of a model neonate, such that a visual comparison between the vital functions of the neonatal test subject and the model neonate is provided.

10. The method of claim 9, further comprising effectuating presentation of a notification when a comparison of the test dataset and the selected model datasets indicates that the neonatal test subject breaches a development threshold from the model neonate.

11. A system for providing an indication of the relative condition of neonatal subjects, system comprising:
means for obtaining a test dataset which describes monitored vital functions of a neonatal test subject;
means for comparing the test dataset with one or more previously obtained model datasets describing vital functions of neonatal subjects;
means for selecting, based on the comparison, individual ones of the one or more model datasets that are similar to the test dataset;
means for determining, based on the test dataset and the selected model datasets, a progress prediction for the future development of the neonatal test subject; and,
means for effectuating presentation of an indication of the progress prediction.

12. (canceled)

13. The system of claim 11, further comprising:
means for assigning a weighting to the selected similar model datasets based on their closeness to the test dataset; and,
means determining the progress prediction is based on the weighting of the similar model dataset.

14. The system of claim 11, further comprising means for effectuating presentation of an indication of a model dataset describing vital functions of a model neonate, such that a visual comparison between the vital functions of the neonatal test subject and the model neonate is provided.

15. The system of claim 14, further comprising means for effectuating presentation of a notification when a comparison of the test dataset and the selected model datasets indicates that the neonatal test subject breaches a development threshold from the model neonate.
